(19) United States
(12) Patent Application Publication
(10) Pub. No.: US 2026/0256436 A1
(43) Pub. Date: Sep. 3, 2026

(54) RADIATION GENERATION APPARATUS, OPERATION METHOD OF RADIATION GENERATION APPARATUS, AND OPERATION PROGRAM OF RADIATION GENERATION APPARATUS

(71) Applicant: FUJIFILM CORPORATION, Tokyo (JP)

(72) Inventors: Hisatsugu HORIUCHI, Kanagawa (JP); Tatsuya TANEICHI, Kanagawa (JP); Takeyasu KOBAYASHI, Kanagawa (JP); Kyohei MORI, Kanagawa (JP)

(21) Appl. No.: 19/534,755

(22) Filed: Feb. 10, 2026

(30) Foreign Application Priority Data
Feb. 28, 2025 (JP) 2025-031677

(51) Int. Cl. A61B 6/00; A61B 6/42

(52) U.S. Cl. CPC A61B 6/4405; A61B 6/4283

(57) ABSTRACT

A radiation generation apparatus includes a body part having a radiation source that emits radiation toward a patient, the body part being capable of autonomous driving by a carriage unit having wheels. A self-position estimation/map data creation unit estimates a self-position from a driving control image captured by a camera and creates map data of a surrounding environment. The self-position estimation/map data creation unit acquires top plate position detection data and holder position detection data of a top plate position detection sensor and a holder position detection sensor that are provided on a top plate of a decubitus imaging table and a holder of an upright imaging table and that detect positions of the top plate and the holder. The self-position estimation/map data creation unit updates the map data by using the top plate position detection data and the holder position detection data.

### CROSS-REFERENCE TO RELATED APPLICATIONS

The present application claims priority under 35 U.S.C. §119 to Japanese Patent Application No. 2025-031677, filed on February 28, 2025. The above application is hereby expressly incorporated by reference, in its entirety, into the present application.

### BACKGROUND

### 1. Technical Field

The present disclosed technology relates to a radiation generation apparatus, an operation method of a radiation generation apparatus, and an operation program of a radiation generation apparatus.

### 2. Description of the Related Art

In a medical field, a driving type radiation generation apparatus in which a body part having a radiation source that emits radiation toward a subject is capable of driving by a driving mechanism having wheels is used. For example, the driving type radiation generation apparatus is used for so-called ward round imaging in which a patient as a subject is imaged while moving around a ward. In addition, a driving type radiation generation apparatus capable of autonomous driving by Simultaneous Localization and Mapping (SLAM) technology is also being studied.

EP4330913A discloses a device that is a computed tomography (CT) apparatus instead of a radiation generation apparatus but is capable of autonomous driving by SLAM technology. In EP4330913A, map data of a surrounding environment according to output data of a light detection and ranging (LiDAR) sensor mounted on the device is created. Further, in order to supplement information on blind spots or transparent structures that are difficult to detect in the LiDAR sensor, an optical sensor and a radar sensor are mounted on the device, and the map data is updated by using output data of these sensors.

### SUMMARY

In EP4330913A, the optical sensor and the radar sensor are easily affected by disturbances such as movements of an operator such as a medical radiologist, and accurate output data may not be obtained.

One embodiment according to the disclosed technology provides a radiation generation apparatus, an operation method of a radiation generation apparatus, and an operation program of a radiation generation apparatus that can obtain map data reflecting a position and/or a posture of a structure in a surrounding environment while suppressing an influence of a disturbance.

A radiation generation apparatus according to the present disclosed technology is a radiation generation apparatus, in which a body part including a radiation source that emits radiation toward a subject is autonomously drivable by a driving mechanism having wheels, the radiation generation apparatus comprising: an environment information sensor; and a processor, in which the processor is configured to: create map data of a surrounding environment while estimating a self-position from output data of the environment information sensor; acquire detection data of a detection sensor that is provided in a structure in the surrounding environment and that detects a position and/or a posture of a movable component of the structure; and update the map data by using the detection data.

It is preferable that the processor is configured to estimate a driving amount of the driving mechanism based on a change amount of a position of the same structure included in past output data and current output data.

It is preferable that the processor is configured to: recognize the component during movement based on the detection data; and estimate the driving amount by excluding the component recognized as being in movement.

It is preferable that the map data is three-dimensional data.

It is preferable that the map data is set to have a lower spatial resolution in a height direction than in a horizontal direction.

It is preferable that the processor is configured to change a driving speed of the driving mechanism according to a distance to the structure.

It is preferable that the processor is configured to increase a degree of reduction in the driving speed as the distance to the structure is shorter.

It is preferable that the environment information sensor includes a distance sensor, and the output data includes distance data, and the processor is configured to correct the distance data by using a displacement amount of the component based on the detection data.

It is preferable that the structure includes a decubitus imaging table, and the component is a top plate of the decubitus imaging table.

It is preferable that the structure includes an upright imaging table, and the component is a holder of the upright imaging table.

An operation method of a radiation generation apparatus according to the present disclosed technology is an operation method of a radiation generation apparatus, in which a body part including a radiation source that emits radiation toward a subject is autonomously drivable by a driving mechanism having wheels, the operation method comprising: creating map data of a surrounding environment while estimating a self-position from output data of an environment information sensor; acquiring detection data of a detection sensor that is provided in a structure in the surrounding environment and that detects a position and/or a posture of a movable component of the structure; and updating the map data by using the detection data.

An operation program of a radiation generation apparatus according to the present disclosed technology is an operation program of a radiation generation apparatus, in which a body part including a radiation source that emits radiation toward a subject is autonomously drivable by a driving mechanism having wheels, the operation program causing a computer to execute a process comprising: creating map data of a surrounding environment while estimating a self-position from output data of an environment information sensor; acquiring detection data of a detection sensor that is provided in a structure in the surrounding environment and that detects a position and/or a posture of a movable component of the structure; and updating the map data by using the detection data.

According to the disclosed technology, it is possible to provide a radiation generation apparatus, an operation method of a radiation generation apparatus, and an operation program of a radiation generation apparatus that can obtain map data reflecting a structure in a surrounding environment while suppressing an influence of a disturbance.

### BRIEF DESCRIPTION OF THE DRAWINGS

Exemplary embodiments according to the technique of the present disclosure will be described in detail based on the following figures, wherein:

FIG. 1 is a diagram showing a state of decubitus imaging using a radiography system;

FIG. 2 is a diagram showing a state of upright imaging using the radiography system;

FIG. 3 is a block diagram showing an electric configuration of a radiation generation apparatus;

FIG. 4 is a diagram showing a state in which the radiation generation apparatus drives from a standby position toward a first target position in decubitus imaging and stops at the first target position;

FIG. 5 is a diagram showing a state in which the radiation generation apparatus drives from a standby position toward a second target position in upright imaging and stops at the second target position;

FIG. 6 is a diagram showing a process of each processing unit of a processor related to driving control;

FIG. 7 is a diagram showing processing of each processing unit of a processor related to driving control;

FIG. 8 is a diagram showing a state in which a driving amount of a carriage unit is estimated based on a change amount of a position of the same feature point;

FIG. 9 is a diagram showing a state in which a movable component during movement is recognized based on the detection data, and the driving amount is estimated by excluding the movable component recognized as being in movement;

FIG. 10 is a diagram showing a state in which the map data is updated by using top plate position detection data and holder position detection data;

FIG. 11 is a diagram showing a spatial resolution in a horizontal direction and a height direction of the map data;

FIG. 12 is a diagram showing a process of each processing unit of a processor related to alignment control;

FIG. 13 is a diagram showing a process in a learning phase of a cassette contour extraction model;

FIG. 14 is a diagram showing a state of the alignment control;

FIG. 15 is a flowchart showing an imaging procedure;

FIG. 16 is a diagram showing a state in which the driving control is transitioned to the alignment control in a case in which the electronic cassette is detected from the alignment control image;

FIG. 17 is a diagram showing a state in which the driving control is transitioned to the alignment control in a case in which the patient is detected from the alignment control image;

FIG. 18 is a diagram showing an aspect in which a joint point of the patient is extracted from the alignment control image;

FIG. 19 is a table showing an aspect in which a driving speed of the carriage unit is changed according to a distance to the structure;

FIG. 20 is a diagram showing a decubitus imaging table in which a top plate is rotatable; and

FIG. 21 is a diagram showing an aspect in which distance data of the distance sensor is corrected by using a displacement amount of the component based on the detection data.

### DETAILED DESCRIPTION

### First Embodiment

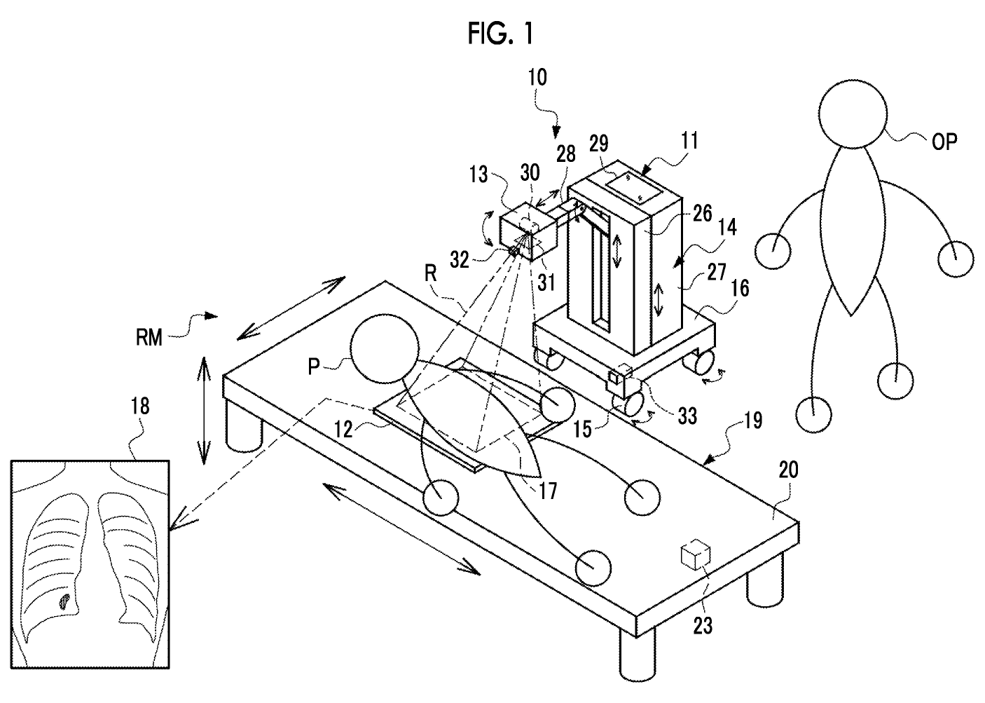

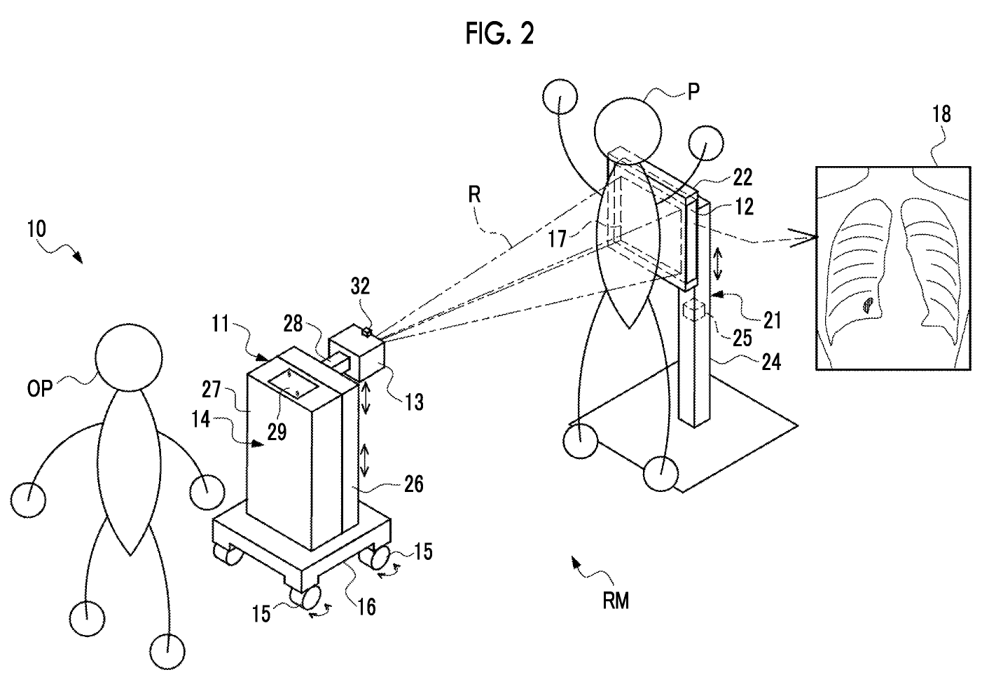

As shown in FIGS. 1 and 2 as an example, a radiography system 10 comprises a radiation generation apparatus 11 and an electronic cassette 12. The radiation generation apparatus 11 has a configuration in which a body part 14 having a radiation source 13 that emits radiation R toward a patient P is mounted on a carriage unit 16 having wheels 15. The radiation R is, for example, X-rays. A battery is mounted in the radiation generation apparatus 11, and the radiation generation apparatus 11 can be driven by being supplied with power from the battery. In addition, the radiation generation apparatus 11 can also be driven while being supplied with power from a commercial power supply via a power supply cord. The carriage unit 16 is an example of a "driving mechanism" according to the disclosed technology. The patient P is an example of a "subject" according to the technology of the present disclosure.

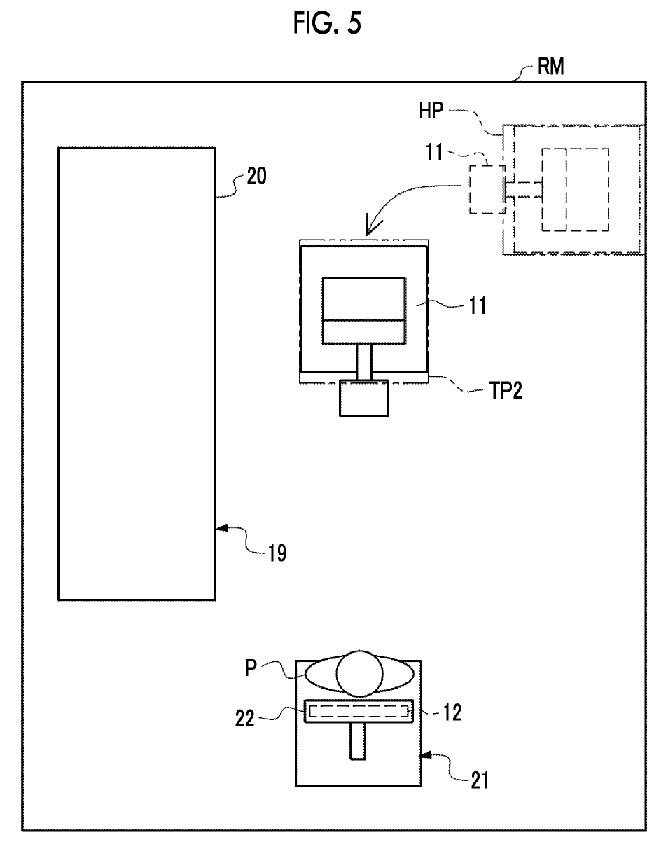

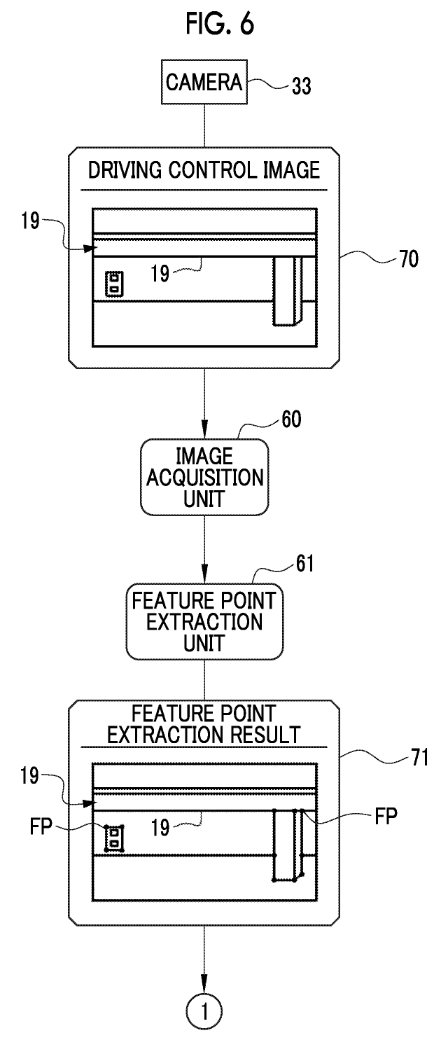

The radiation generation apparatus 11 is movable in an imaging room RM (see also FIGS. 5 and 6). In addition, the radiation generation apparatus 11 is used for so-called ward round imaging in which the patient P is imaged while moving around a ward. Therefore, the radiation generation apparatus 11 is also called a ward round cart. Alternatively, the radiation generation apparatus 11 is also used for imaging in an emergency room. In addition, the radiation generation apparatus 11 can also be carried into an operating room and used during surgery.

The electronic cassette 12 has a configuration in which a sensor panel as a radiation detector that detects the radiation R is built in a portable housing. The electronic cassette 12 is driven by the battery and performs wireless communication with the radiation generation apparatus 11. The sensor panel has a detection surface 17 in which a plurality of pixels that generate signal charges in response to the radiation R or visible light converted from the radiation R by a scintillator are arranged in a matrix. The electronic cassette 12 detects the radiation R emitted from the radiation source 13 and transmitted through the patient P, and outputs a radiographic image 18 of the patient P.

Since the electronic cassette 12 is portable and wireless, as shown in FIG. 1, the electronic cassette 12 can be used for so-called free imaging in which the electronic cassette 12 is placed on a top plate 20 of a decubitus imaging table (bed) 19 installed in the imaging room RM (under the patient P) to perform radiography. More specifically, FIG. 1 shows a state in which the electronic cassette 12 is inserted between the top plate 20 and the patient P to image the patient P lying on the top plate 20 (see also FIG. 5). In addition, as shown in FIG. 2, the electronic cassette 12 can also be used by being accommodated in a holder 22 of an upright imaging table 21 installed in the imaging room RM (see also FIG. 6).

The top plate 20 of the decubitus imaging table 19 is movable in the front-rear-left-right direction. In addition, the top plate 20 is movable in the up-down direction. A top plate position detection sensor 23 is provided on the top plate 20. The top plate position detection sensor 23 detects a horizontal position and a height position of the top plate 20. The top plate position detection sensor 23 is, for example, a rotary encoder, a potentiometer, a gyro sensor, or a combination of a plurality of types of these sensors. The decubitus imaging table 19 is an example of a "structure" according to the disclosed technology. The top plate 20 is an example of a "component" according to the disclosed technology. In addition, the top plate position detection sensor 23 is an example of a "detection sensor" according to the disclosed technology.

The holder 22 of the upright imaging table 21 is movable in the up-down direction with respect to a support column 24. A holder position detection sensor 25 is provided on the support column 24. The holder position detection sensor 25 detects a height position of the holder 22. The holder position detection sensor 25 is, for example, a rotary encoder, a potentiometer, a gyro sensor, or a combination of a plurality of types of these sensors. The upright imaging table 21 is an example of a "structure" according to the disclosed technology. The holder 22 is an example of a "component" according to the disclosed technology. In addition, the holder position detection sensor 25 is an example of a "detection sensor" according to the disclosed technology.

The body part 14 has a rectangular parallelepiped shape and is erected at the center of the carriage unit 16. The body part 14 is divided into a front part 26 and a rear part 27. The front part 26 is movable in the up-down direction with respect to the rear part 27. The rear part 27 is fixed to the carriage unit 16.

A base end of an arm 28 is attached to the front part 26. More specifically, the arm 28 is divided into a first portion in which the base end is attached to the front part 26 and a second portion in which the base end is attached to the first portion. The radiation source 13 is attached to a distal end of the second portion that is a free end opposite to the base end.

The first portion is movable in the up-down direction with respect to the front part 26 and is bendable in the up-down direction with respect to the front part 26. The second portion is bendable up and down with respect to the first portion. Further, the second portion is expandable and contractible. The radiation source 13 is rotatable with respect to the second portion, that is, is swingable. By the displacement of the arm 28 and the displacement of the radiation source 13 with respect to the arm 28, a height position, a horizontal position, and a posture (orientation) of the radiation source 13 can be adjusted.

An operation panel 29 is provided on an upper surface of the rear part 27. The operation panel 29 is configured by, for example, a touch panel display, and has a function of displaying information in addition to an operation function. The operation panel 29 is operated by an operator OP such as a medical radiologist. The operator OP sets an irradiation condition of the radiation R through the operation panel 29. In addition, the operator OP checks the radiographic image 18 through the operation panel 29.

In addition, an irradiation switch (not shown) is provided in the rear part 27. The irradiation switch is a switch that is provided to allow the operator OP to give an instruction to start irradiation of radiation. An extension cable is connected to the irradiation switch, and can be detached from the rear part 27 for use. The irradiation switch is operable only after driving control and alignment control, which will be described below, are completed.

The radiation source 13 includes a radiation tube 30 and an irradiation field limiter 31. The radiation tube 30 generates the radiation R. The radiation tube 30 is provided with a filament, a target, a grid electrode, and the like (all are not illustrated). A tube voltage is applied between the filament that is a cathode and the target that is an anode from a voltage generator (not shown) built in the rear part 27. The filament releases thermal electrons according to the applied tube voltage toward the target. The target radiates the radiation R with collision of the thermoelectrons released from the filament. The grid electrode is disposed between the filament and the target, and changes a flow rate of the thermoelectrons from the filament toward the target in response to the voltage applied from the voltage generator. The flow rate of the thermal electrons from the filament toward the target is referred to as a tube current. The tube voltage and the tube current are set to the radiation source 13 as the irradiation condition as the irradiation time elapses.

The irradiation field limiter 31 is also called a collimator and limits an irradiation field of the radiation R generated from the radiation tube 30. For example, the irradiation field limiter 31 has a configuration in which four shield plates formed of lead or the like shielding radiation R are disposed on respective sides of a quadrangle, and an emission opening of the quadrangle transmitting radiation is formed in a center portion. The irradiation field limiter 31 changes a size of the emission opening by changing a position of each shielding plate, thereby changing the irradiation field of the radiation R.

A camera 32 is attached to the radiation source 13. The camera 32 is used to support the alignment between the radiation source 13 and the electronic cassette 12. The camera 32 incorporates an imaging element that is sensitive to visible light, for example, a complementary metal oxide semiconductor (CMOS) image sensor or a charge-coupled device (CCD) image sensor. The camera 32 captures a video image at a predetermined frame rate.

The wheels 15 are provided four in total in front, rear, left, and right of the carriage unit 16. That is, the carriage unit 16 is a four-wheel type. Each wheel 15 is, for example, a revolution type that revolves around a revolution axis extending in a height direction (also referred to as a vertical direction) orthogonal to a rotation axis in a case of driving and rotating. The body part 14 autonomously drives by the carriage unit 16.

Here, the autonomous driving refers to autonomously driving toward a set target position while estimating a self-position while recognizing a surrounding environment by using the environment information sensor. For example, a position on a side of the decubitus imaging table 19 shown in FIG. 1 and a position facing the upright imaging table 21 shown in FIG. 2 are set in advance as the target position. In the radiation generation apparatus 11, the SLAM technology is used to implement the autonomous driving.

A camera 33 is provided in the carriage unit 16. The camera 33 is used to support the autonomous driving of the body part 14. The camera 33 images a front side of the carriage unit 16. The camera 33 also incorporates an imaging element that is sensitive to visible light and captures a video image at a predetermined frame rate, as in the camera 32. The camera 33 is an example of an "environment information sensor" according to the disclosed technology. The body part 14 and the radiation generation apparatus 11 can also be manually drived by the operator OP, in addition to the autonomous driving.

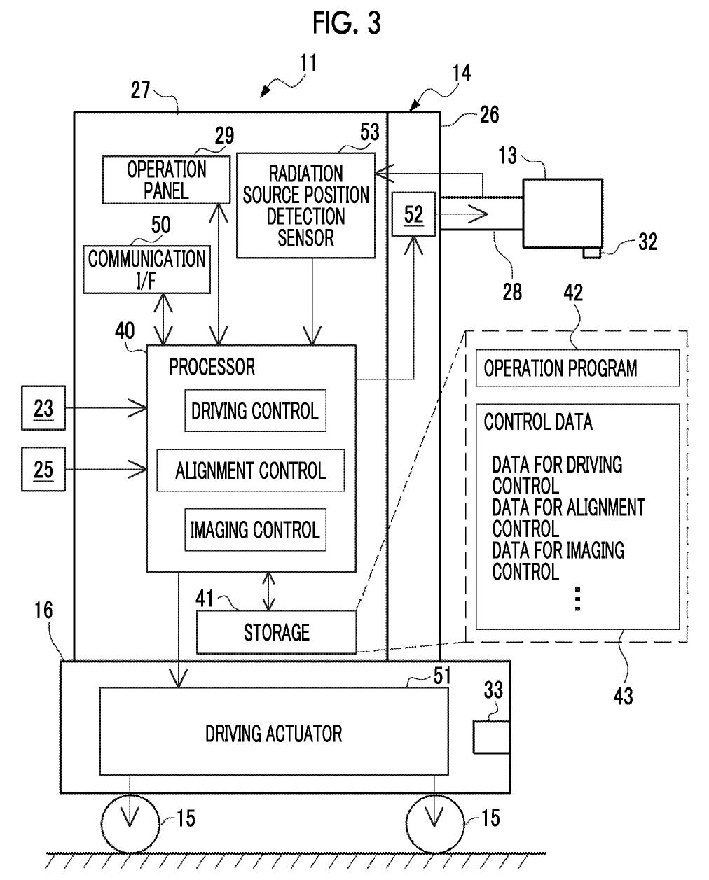

FIG. 3 is a block diagram showing an example of an electric configuration of the radiation generation apparatus 11. A processor 40 integrally controls the entire radiation generation apparatus 11. The processor 40 is configured by, for example, a central processing unit (CPU) and a memory such as a random access memory (RAM), and functions as various processing units by loading and executing various programs in the memory. Specifically, the processor 40 executes driving control, alignment control, and imaging control. The driving control is control related to the autonomous driving of the body part 14. The alignment control is control related to the alignment between the radiation source 13 and the electronic cassette 12. The imaging control includes irradiation control of the radiation R by the radiation source 13 and output control of the radiographic image 18 by the electronic cassette 12.

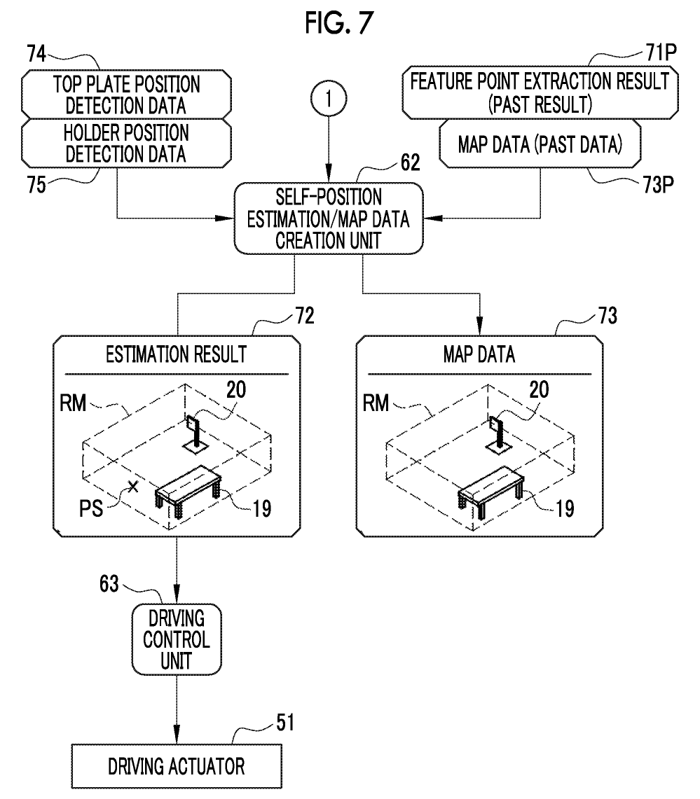

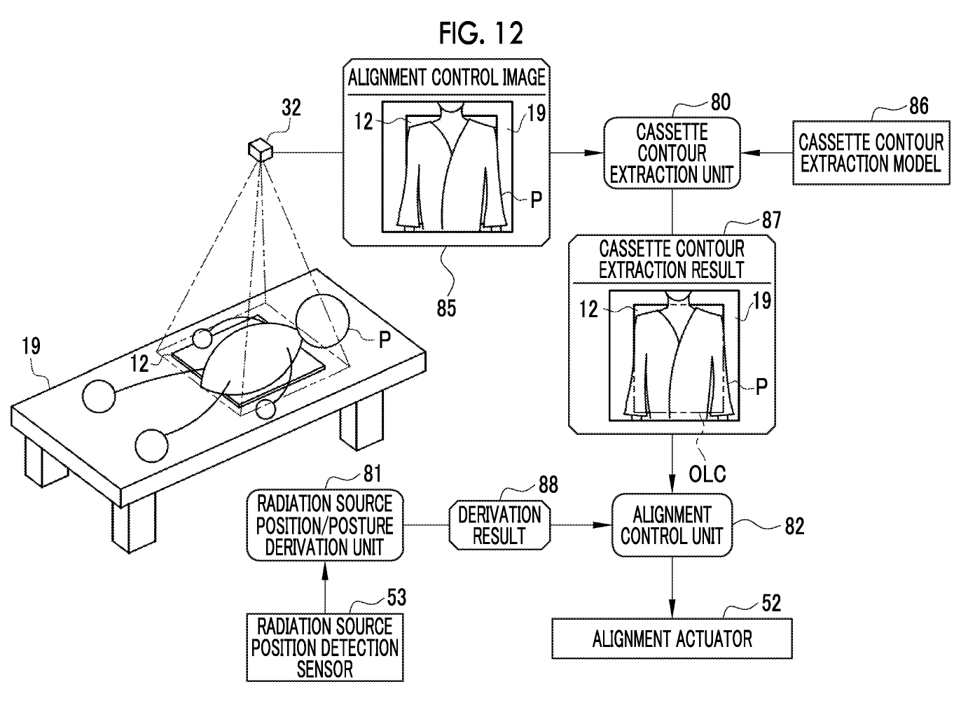

A storage 41 is configured by a non-volatile memory such as a hard disk drive or a solid state drive. The storage 41 stores an operation program 42 and control data 43. The operation program 42 is an example of an "operation program of a radiation generation apparatus" according to the disclosed technology. The control data 43 includes data for driving control, data for alignment control, and data for imaging control. Examples of the data for driving control include map data 73 (see FIG. 7) created by the SLAM technology. Examples of the data for alignment control include a cassette contour extraction model 86 (see FIG. 12) for extracting a contour OLC (see FIG. 12) of the electronic cassette 12. Examples of the data for imaging control include an irradiation condition table in which an irradiation condition for each imaging part is registered.

The processor 40 is connected to the top plate position detection sensor 23, the holder position detection sensor 25, the operation panel 29, a communication interface (I/F) 50, a driving actuator 51, an alignment actuator 52, and a radiation source position detection sensor 53. The processor 40 receives top plate position detection data 74 (see FIG. 7) from the top plate position detection sensor 23 and receives holder position detection data 75 (see FIG. 7) from the holder position detection sensor 25. The processor 40 updates the map data 73 (see FIG. 7) of the surrounding environment by using the top plate position detection data 74 and the holder position detection data 75.

The processor 40 performs display control of various screens on the operation panel 29. In addition, the processor 40 receives various operation instructions of the operator OP through the operation panel 29 and executes various controls in response to the various operation instructions. The communication I/F 50 is, for example, a wireless communication I/F and performs wireless communication with the electronic cassette 12.

The driving actuator 51 includes a motor for driving rotation of the wheels 15 and a motor for revolution under the control of the processor 40. The alignment actuator 52 includes a motor for elevating the front part 26, a motor for elevating the arm 28, a motor for bending the second portion of the arm 28, a motor for expanding and contracting the second portion, and a motor for rotating the radiation source 13 with respect to the second portion, under the control of the processor 40.

The radiation source position detection sensor 53 measures an elevation direction and an elevation amount of the arm 28 with respect to the front part 26, a bending direction and a bending amount of the second portion of the arm 28 with respect to the first portion, an expansion and contraction direction and an expansion and contraction amount of the second portion, and a rotation direction and a rotation amount of the radiation source 13 with respect to the second portion. The radiation source position detection sensor 53 is, for example, a rotary encoder, a potentiometer, a gyro sensor, or a combination of a plurality of types of these sensors. The radiation source position detection sensor 53 outputs the measurement value to the processor 40. The processor 40 derives a position and a posture of the radiation source 13 based on the measurement value of the radiation source position detection sensor 53.

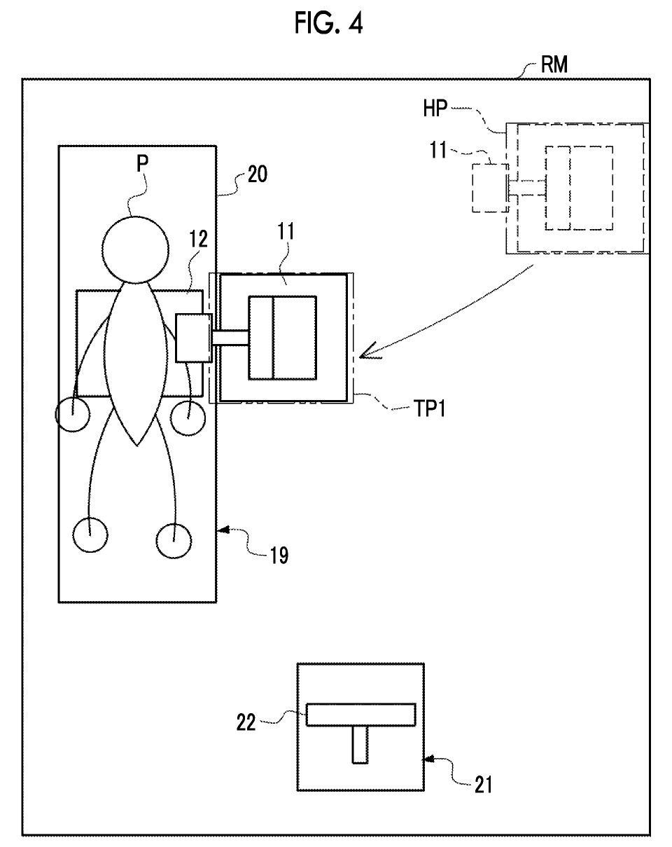

As shown in FIGS. 4 and 5 as an example, a standby position HP of the radiation generation apparatus 11 is prepared in a corner of the imaging room RM. In the standby position HP, charging of the battery, receiving of an imaging order from a radiology information system (RIS), setting of the irradiation condition, and the like are performed. The standby position HP occupies a region equal to, or one size larger than, that of the radiation generation apparatus 11.

In the imaging room RM, a first target position TP1 (see FIG. 4) of the radiation generation apparatus 11 in the decubitus imaging and a second target position TP2 (see FIG. 5) of the radiation generation apparatus 11 in the upright imaging are set. The first target position TP1 is a position at a side of the decubitus imaging table 19, and more specifically, a position facing one long side of the decubitus imaging table 19. The second target position TP2 is a position facing the upright imaging table 21 and separated from the upright imaging table 21 by a distance of an SID required for the upright imaging. The first target position TP1 and the second target position TP2 occupy a region equal to, or one size larger than, that of the radiation generation apparatus 11, like the standby position HP. In the following, the first target position TP1 and the second target position TP2 may be collectively referred to as a target position TP.

In a case of the decubitus imaging, the radiation generation apparatus 11 drives from the standby position HP toward the first target position TP1 and stops in a case in which the self-position PS (see FIG. 7) reaches the first target position TP1 (see FIG. 4). In addition, in a case of the upright imaging, the radiation generation apparatus 11 drives from the standby position HP toward the second target position TP2 and stops in a case in which the self-position PS reaches the second target position TP2 (see FIG. 5). Although not shown, the radiation generation apparatus 11 may drive from the first target position TP1 toward the second target position TP2 in order to perform the upright imaging after the decubitus imaging. In addition, the radiation generation apparatus 11 may drive from the second target position TP2 toward the first target position TP1 in order to perform the decubitus imaging after the upright imaging. In a case where the self-position PS matches the center of the target position TP, it may be determined that the self-position PS has reached the target position TP, or in a case where the self-position PS is within a range including the center of the target position TP with a certain margin, it may be determined that the self-position PS has reached the target position TP.

The autonomous driving to the target position TP is started by, for example, an instruction of the operator OP through the operation panel 29. In addition to or instead of the operation panel 29, a configuration may be adopted in which an instruction to start the autonomous driving can be issued by a remote controller.

The processor 40 executes the driving control using the SLAM technology. Specifically, as shown in FIGS. 6 and 7, the processor 40 functions as an image acquisition unit 60, a feature point extraction unit 61, a self-position estimation/map data creation unit 62, and a driving control unit 63.

The image acquisition unit 60 sequentially acquires an image 70 (hereinafter, referred to as a driving control image) of the surrounding environment of the radiation generation apparatus 11 captured by the camera 33 at a predetermined frame rate. The image acquisition unit 60 performs preprocessing such as noise removal and distortion correction on the driving control image 70 and then outputs the driving control image 70 to the feature point extraction unit 61. The driving control image 70 is an example of "output data" according to the disclosed technology.

The feature point extraction unit 61 extracts a corner of a structure present in the surrounding environment shown in the driving control image 70 as a feature point FP by using an algorithm such as oriented features from accelerated segment test and rotated binary robust independent elementary features (ORB) or speeded-up robust features (SURF). The feature point extraction unit 61 outputs a feature point extraction result 71 to the self-position estimation/map data creation unit 62. In addition, although not shown, the feature point extraction unit 61 stores the feature point extraction result 71 in the storage 41. The feature point extraction result 71 is a set of coordinates of each feature point FP and a feature amount vector.

The self-position estimation/map data creation unit 62 estimates the self-position PS of the radiation generation apparatus 11 and creates map data 73 of the surrounding environment. The feature point extraction result 71 is input to the self-position estimation/map data creation unit 62 from the feature point extraction unit 61. In addition, a feature point extraction result (hereinafter, referred to as a feature point extraction result (past result)) 71P for a plurality of past frames and map data (hereinafter, referred to as map data (past data)) 73P for a plurality of past frames are input to the self-position estimation/map data creation unit 62. Further, the top plate position detection data 74 from the top plate position detection sensor 23 and the holder position detection data 75 from the holder position detection sensor 25 are input to the self-position estimation/map data creation unit 62. The top plate position detection data 74 and the holder position detection data 75 are examples of "detection data" according to the disclosed technology.

The feature point extraction result (past result) 71P and the map data (past data) 73P are stored in the storage 41 as the data for driving control of the control data 43. The feature point extraction result 71 and the map data 73 constituting the feature point extraction result (past result) 71P and the map data (past data) 73P are, for example, for several tens to several hundreds of frames. The feature point extraction result (past result) 71P and the map data (past data) 73P include the feature point extraction result 71 and the map data 73 that are considered to play an important role in the estimation of the self-position PS and the creation of the map data 73. The feature point extraction result 71 and the map data 73 are, for example, the feature point extraction result 71 and the map data 73 obtained for each movement of a certain distance. In addition, for example, the feature point extraction result 71 and the map data 73 obtained in a case where a large viewpoint change has occurred from the previous frame. Further, for example, the feature point extraction result 71 and the map data 73 obtained in a case where a set amount or more of new feature points FP are extracted.

The self-position estimation/map data creation unit 62 matches the feature point FP of the feature point extraction result 71 from the feature point extraction unit 61 with the feature point FP of the feature point extraction result (past result) 71P. In this case, the self-position estimation/map data creation unit 62 refers to the feature amount vector of each feature point FP. More specifically, the feature point FP having a distance (Euclidean distance or the like) of the feature amount vector less than a threshold value between the feature point FP of the feature point extraction result 71 from the feature point extraction unit 61 and the feature point FP of the feature point extraction result (past result) 71P is recognized as the same feature point FP. The self-position estimation/map data creation unit 62 estimates the self-position PS based on the matching result of the feature point FP and the map data (past data) 73P.

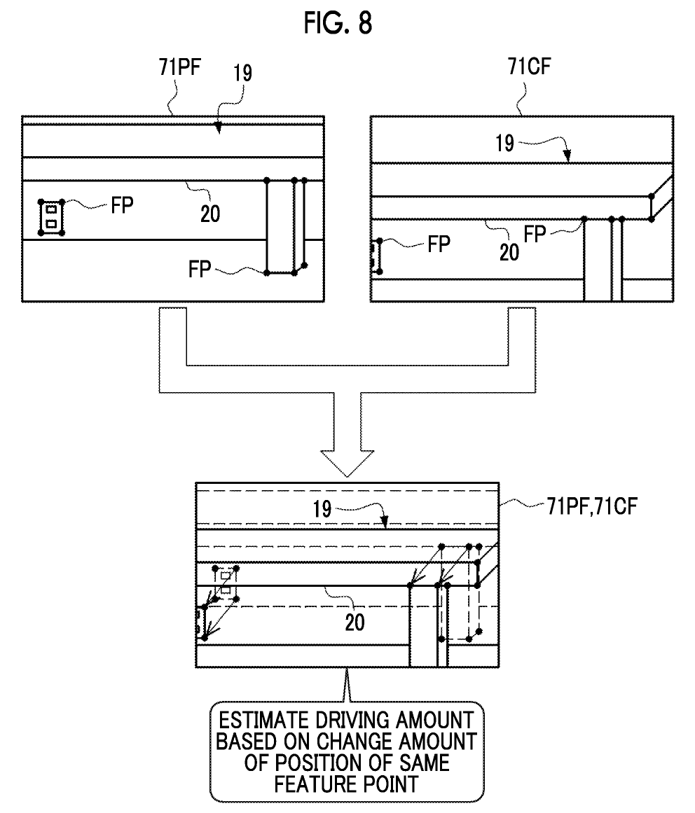

As shown in FIG. 8 as an example, the self-position estimation/map data creation unit 62 estimates the driving amount of the carriage unit 16 based on a change amount of the position of the same feature point FP in a feature point extraction result 71PF of the previous frame and a feature point extraction result 71CF of the current frame. Then, the self-position PS is estimated with reference to the estimation result of the driving amount of the carriage unit 16. The feature point extraction result 71PF of the previous frame is an example of "past output data" according to the disclosed technology. The feature point extraction result 71CF of the current frame is an example of "current output data" according to the disclosed technology.

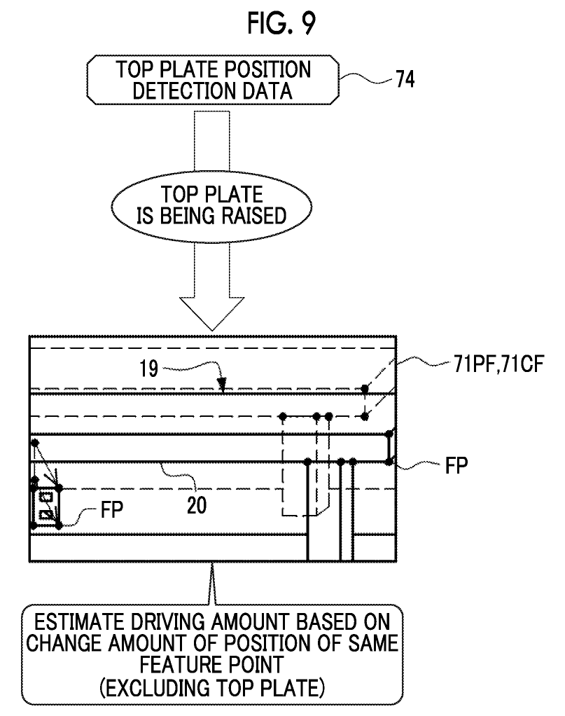

However, as shown in FIG. 9 as an example, the self-position estimation/map data creation unit 62 recognizes the movable component during movement based on the detection data, and estimates the driving amount of the carriage unit 16 by excluding the feature point FP related to the movable component recognized as being in movement. FIG. 9 shows an example in which it is recognized that the top plate 20 is being raised based on the top plate position detection data 74, and the driving amount of the carriage unit 16 is estimated by excluding the feature point FP related to the top plate 20. The determination as to whether or not the feature point FP is the feature point FP related to the component is performed, for example, based on an image obtained by inputting the driving control image 70 to a semantic segmentation model and in which each structure and each component shown in the driving control image 70 are distinguished.

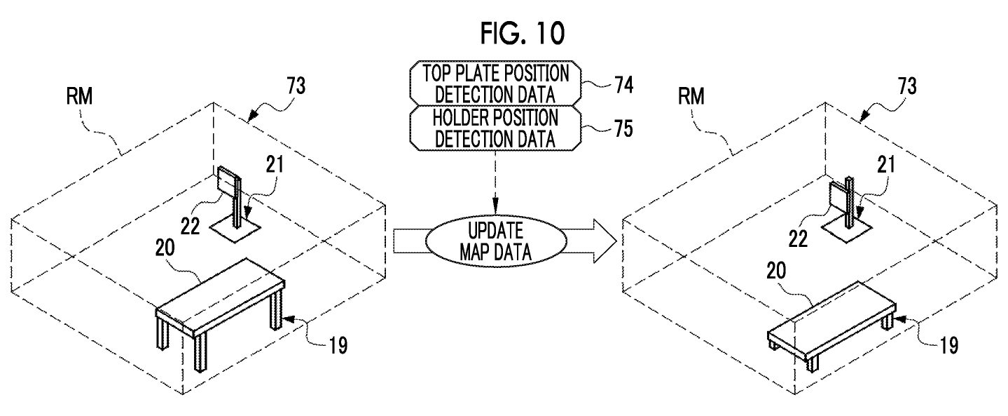

The self-position estimation/map data creation unit 62 creates (updates the map data 73) new map data 73 based on the feature point extraction result 71 from the feature point extraction unit 61, the feature point extraction result (past result) 71P, the map data (past data) 73P, and an estimation result 72. In this case, as shown in FIG. 10 as an example, the self-position estimation/map data creation unit 62 further updates the map data 73 by using the top plate position detection data 74 and the holder position detection data 75. More specifically, the self-position estimation/map data creation unit 62 changes the horizontal position and the height position of the top plate 20 in the map data 73 according to the horizontal position and the height position of the top plate 20 represented by the top plate position detection data 74. In addition, the self-position estimation/map data creation unit 62 changes the height position of the holder 22 in the map data 73 according to the height position of the holder 22 represented by the holder position detection data 75. FIG. 10 shows a case in which the height positions of the top plate 20 and the holder 22 are changed. As described above, the self-position estimation/map data creation unit 62 estimates the self-position PS and creates the map data 73 in parallel in a process in which the radiation generation apparatus 11 autonomously drives. The self-position estimation/map data creation unit 62 does not estimate the self-position PS until a sufficient amount of the feature point extraction result (past result) 71P is accumulated, and only creates (updates) the map data 73.

The self-position estimation/map data creation unit 62 outputs the estimation result 72 of the self-position PS to the driving control unit 63. The driving control unit 63 controls the driving of the driving actuator 51 such that the self-position PS is the target position TP. In addition, although not shown, the self-position estimation/map data creation unit 62 stores the map data 73 in the storage 41.

Specifically, the map data 73 is three-dimensional data of the imaging room RM including the structure such as the decubitus imaging table 19 and the upright imaging table 21. In addition, the standby position HP and the target position TP are registered in the map data 73.

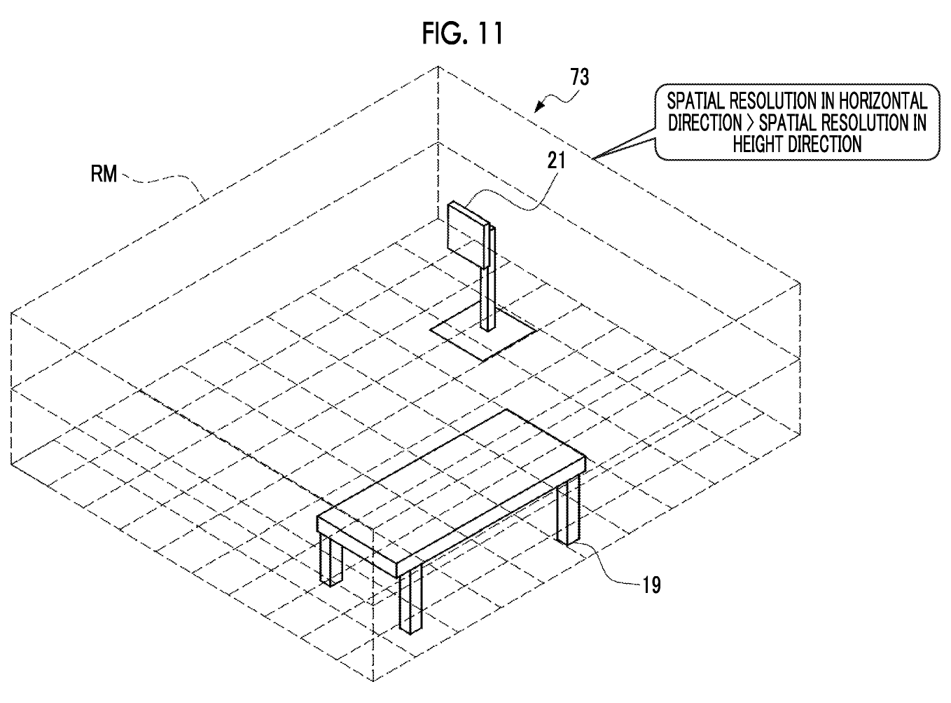

As shown in FIG. 11 as an example, the map data 73 is set to have a lower spatial resolution in the height direction than in the horizontal direction. For example, the spatial resolution in the horizontal direction is 5 cm, and the spatial resolution in the height direction is 10 times 50 cm.

In a case in which the driving actuator 51 is driven under the control of the driving control unit 63 and the self-position PS of the radiation generation apparatus 11 is the target position TP, the processor 40 transitions from the driving control to the alignment control.

As shown in FIG. 12 as an example, the processor 40 functions as a cassette contour extraction unit 80, a radiation source position/posture derivation unit 81, and an alignment control unit 82.

An image 85 (hereinafter, referred to as an alignment control image) including the patient P and the electronic cassette 12 captured by the camera 32 at a predetermined frame rate is sequentially input to the cassette contour extraction unit 80. In a case where the self-position PS of the radiation generation apparatus 11 is the target position TP, the patient P and the electronic cassette 12 are shown in the alignment control image 85. FIG. 12 shows the decubitus imaging, so that the decubitus imaging table 19 is also shown in the alignment control image 85. It is assumed that the alignment between the patient P and the electronic cassette 12 is performed by the operator OP before the driving control and the alignment control.

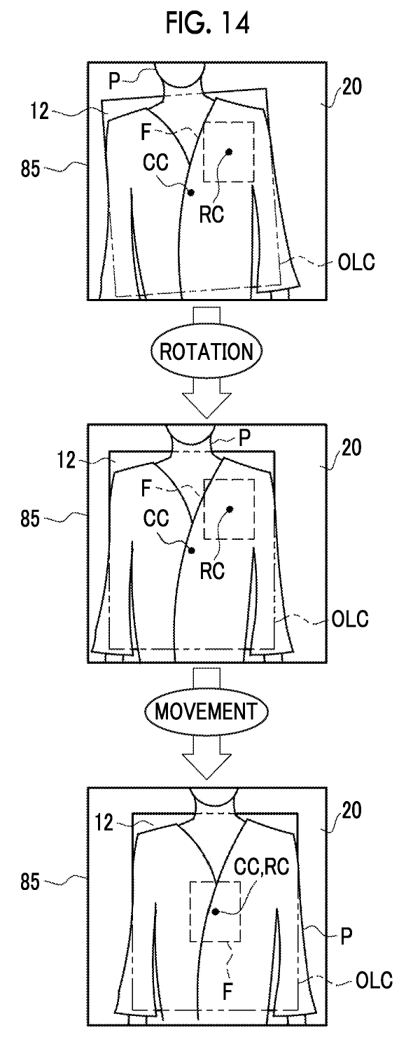

The cassette contour extraction unit 80 extracts the contour OLC of the electronic cassette 12 from the alignment control image 85 by using the cassette contour extraction model 86. According to the contour OLC, a center CC (see FIG. 14) of the detection surface 17 of the electronic cassette 12 and a posture of the electronic cassette 12 with respect to the radiation source 13 are known. The cassette contour extraction unit 80 outputs a cassette contour extraction result 87 to the alignment control unit 82. Here, a contour following all sides of the electronic cassette 12 is illustrated as the contour OLC, but the present disclosure is not limited thereto. Four corners of the electronic cassette 12 may be extracted as the contour.

The cassette contour extraction model 86 is a trained model that is configured by, for example, a convolutional neural network and is trained to output the cassette contour extraction result 87 in a case where the alignment control image 85 is input. The cassette contour extraction model 86 is stored in the storage 41 as the data for alignment control of the control data 43.

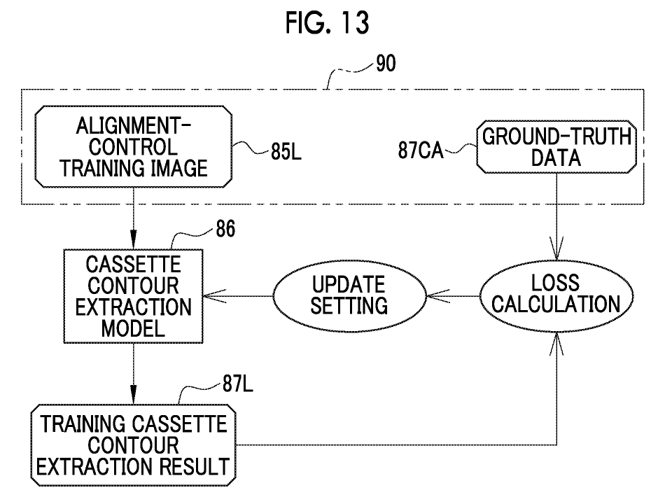

As shown in FIG. 13 as an example, in the learning phase, learning data 90 is given to the cassette contour extraction model 86. The learning data 90 is composed of a set of a alignment-control training image 85L and ground-truth data 87CA. The electronic cassette 12 is shown in the alignment-control training image 85L. The ground-truth data 87CA is data in which the contour OLC of the electronic cassette 12 shown in the alignment-control training image 85L is annotated, and is data for checking the answer.

The alignment-control training image 85L is input to the cassette contour extraction model 86. The cassette contour extraction model 86 outputs a training cassette contour extraction result 87L in response to the input of the alignment-control training image 85L. A loss calculation of the cassette contour extraction model 86 using a loss function is performed based on the training cassette contour extraction result 87L and the ground-truth data 87CA. Then, update settings of various coefficients (coefficients of filters of a convolutional layer and the like) of the cassette contour extraction model 86 are made according to the result of the loss calculation, and the cassette contour extraction model 86 is updated according to the update settings.

In the learning phase of the cassette contour extraction model 86, the series of processing of the input of the alignment-control training image 85L to the cassette contour extraction model 86, the output of the training cassette contour extraction result 87L from the cassette contour extraction model 86, the loss calculation, the update setting, and the update of the cassette contour extraction model 86 is repeatedly performed while the learning data 90 is replaced. The repetition of the series of processing is ended in a case where the extraction accuracy of the training cassette contour extraction result 87L reaches a predetermined set level. The cassette contour extraction model 86 in which the extraction accuracy reaches the set level is stored in the storage 41 and is used by the cassette contour extraction unit 80. The learning may be ended in a case where the series of processing is repeated a set number of times regardless of the extraction accuracy of the training cassette contour extraction result 87L.

The radiation source position/posture derivation unit 81 derives the position and the posture of the radiation source 13 based on the measurement value of the radiation source position detection sensor 53. The radiation source position/posture derivation unit 81 outputs a derivation result 88 to the alignment control unit 82. The derivation result 88 is coordinates of an irradiation center RC (see FIG. 14) of the radiation R in the alignment control image 85 and coordinates of a rectangular frame F (see FIG. 14) indicating the posture of the radiation source 13 in the alignment control image 85. Since the positional relationship between the radiation source 13 and the camera 32 is known, the coordinates of the irradiation center RC and the frame F in the alignment control image 85 can be easily calculated from the measurement value of the radiation source position detection sensor 53.

The alignment control unit 82 controls the driving of the alignment actuator 52 such that the radiation source 13 and the electronic cassette 12 face each other. More specifically, as shown in FIG. 14 as an example, in a case in which the posture of the radiation source 13 is inclined with respect to the electronic cassette 12, the alignment control unit 82 rotates the radiation source 13 to eliminate the inclination. In addition, in a case where the irradiation center RC of the radiation R and the center CC of the detection surface 17 of the electronic cassette 12 are shifted, the alignment control unit 82 moves the radiation source 13 to eliminate the shift. After the shift between the irradiation center RC of the radiation R and the center CC of the detection surface 17 of the electronic cassette 12 is eliminated, the inclination may be eliminated. In addition, FIG. 14 shows a case in which the posture of the radiation source 13 is inclined around the normal line of the detection surface 17, but the present disclosure is not limited to this. Even in a case where the posture of the radiation source 13 is inclined around an axis along the long side of the detection surface 17 or around an axis along the short side of the detection surface 17, the alignment control unit 82 rotates the radiation source 13 to eliminate the inclination.

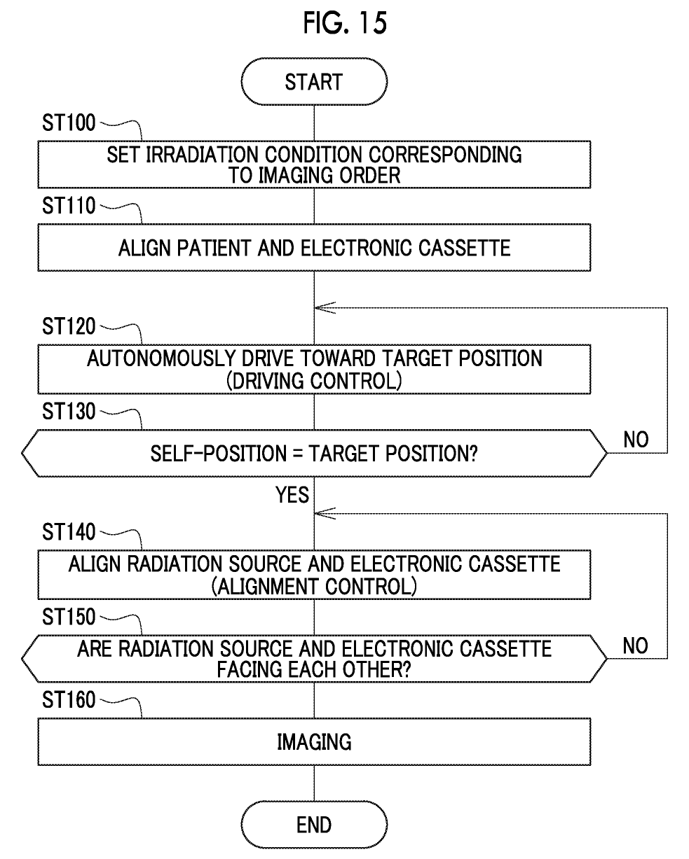

Next, an operation of the configuration described above will be described with reference to the flowchart shown in FIG. 15 as an example. Before the imaging, the radiation generation apparatus 11 waits at the standby position HP in the imaging room RM.

An imaging order is transmitted from the radiology information system to the radiation generation apparatus 11. The operator OP operates the operation panel 29 to set the irradiation condition corresponding to the imaging order (step ST100).

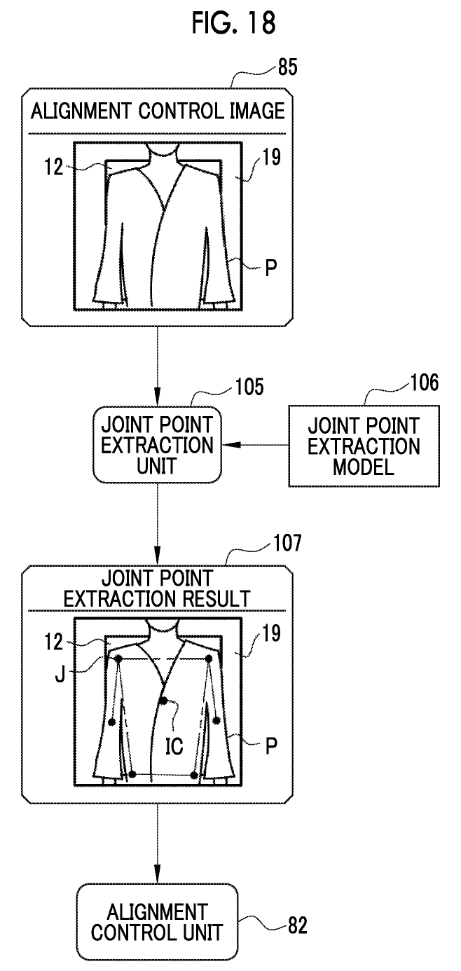

The operator OP places the electronic cassette 12 on the top plate 20 of the decubitus imaging table 19 in a case of the decubitus imaging, and accommodates the electronic cassette 12 in the holder 22 of the upright imaging table 21 in a case of the upright imaging. Then, the alignment between the patient P and the electronic cassette 12 is performed (step ST110). Specifically, the center IC (see FIG. 18) of the imaging part of the patient P and the center CC of the detection surface 17 of the electronic cassette 12 are aligned. In addition, the body axis (head-tail axis) of the patient P and the long side of the detection surface 17 of the electronic cassette 12 are made parallel to each other.

The autonomous driving of the radiation generation apparatus 11 from the standby position HP toward the target position TP is started in response to the instruction of the operator OP (step ST120). In the autonomous driving, the processor 40 executes the driving control using the SLAM technology, which is shown in FIGS. 6 and 7 as an example.

Specifically, first, the driving control image 70 captured by the camera 33 is acquired by the image acquisition unit 60. The driving control image 70 is output from the image acquisition unit 60 to the feature point extraction unit 61. Then, in the feature point extraction unit 61, the feature point FP of the structure present in the surrounding environment shown in the driving control image 70 is extracted. The feature point extraction result 71 is output from the feature point extraction unit 61 to the self-position estimation/map data creation unit 62. In addition, the feature point extraction result 71 is stored in the storage 41.

In the self-position estimation/map data creation unit 62, the self-position PS of the radiation generation apparatus 11 is estimated based on the feature point extraction result 71, the feature point extraction result (past result) 71P, and the map data (past data) 73P, and the estimation result 72 is output. In this case, as shown in FIG. 8, in the self-position estimation/map data creation unit 62, the driving amount of the carriage unit 16 is estimated based on the change amount of the position of the same feature point FP in the feature point extraction result 71PF of the previous frame and the feature point extraction result 71CF of the current frame, and the self-position PS is also estimated with reference to the driving amount. However, the driving amount is estimated by excluding the feature point FP related to the top plate 20 or the holder 22 recognized as being in movement. The estimation result 72 of the self-position PS is output from the self-position estimation/map data creation unit 62 to the driving control unit 63.

In addition, in the self-position estimation/map data creation unit 62, the map data 73 is created based on the feature point extraction result 71, the feature point extraction result (past result) 71P, the map data (past data) 73P, and the estimation result 72. In this case, as shown in FIG. 10, in the self-position estimation/map data creation unit 62, the map data 73 is further updated by using the top plate position detection data 74 and the holder position detection data 75. The map data 73 is stored in the storage 41.

Under the control of the driving control unit 63, the driving of the driving actuator 51 is controlled such that the self-position PS is the target position TP. The driving control is executed in this way. The driving control is continued until the self-position PS of the radiation generation apparatus 11 is the target position TP (NO in step ST130).

In a case in which the self-position PS of the radiation generation apparatus 11 is the target position TP (YES in step ST130), the driving control is transitioned to the alignment control (step ST140).

As shown in FIG. 12, the alignment control image 85 captured by the camera 32 is input to the cassette contour extraction unit 80. In the cassette contour extraction unit 80, the contour OLC of the electronic cassette 12 is extracted from the alignment control image 85 by using the cassette contour extraction model 86. The cassette contour extraction result 87 is output from the cassette contour extraction unit 80 to the alignment control unit 82.

The radiation source position/posture derivation unit 81 derives the position and the posture of the radiation source 13 based on the measurement value of the radiation source position detection sensor 53. The derivation result 88 of the position and the posture of the radiation source 13 is output from the radiation source position/posture derivation unit 81 to the alignment control unit 82.

The driving of the alignment actuator 52 is controlled under the control of the alignment control unit 82 such that the radiation source 13 and the electronic cassette 12 face each other. The alignment control is executed in this way. The alignment control is continued until the radiation source 13 and the electronic cassette 12 face each other (NO in step ST150).

In a case in which the radiation source 13 and the electronic cassette 12 face each other (YES in step ST150), the irradiation switch can be operated. The operator OP operates the irradiation switch to issue an instruction to start the irradiation of the radiation. As a result, the radiation R is emitted from the radiation source 13, and the radiographic image 18 is output by the electronic cassette 12 (step ST160).

As described above, in the radiation generation apparatus 11, the body part 14 having the radiation source 13 that emits the radiation R toward the patient P can autonomously drive by the carriage unit 16 having the wheels 15. The self-position estimation/map data creation unit 62 estimates the self-position PS from the driving control image 70 captured by the camera 33 and creates the map data 73 of the surrounding environment. The self-position estimation/map data creation unit 62 acquires the top plate position detection data 74 and the holder position detection data 75 of the top plate position detection sensor 23 and the holder position detection sensor 25 that are provided on the top plate 20 of the decubitus imaging table 19 and the holder 22 of the upright imaging table 21 and that detect the positions of the top plate 20 and the holder 22. The self-position estimation/map data creation unit 62 updates the map data 73 by using the top plate position detection data 74 and the holder position detection data 75.

Therefore, as in the optical sensor and the radar sensor described in EP4330913A, the positions of the top plate 20 and the holder 22 can be accurately determined without being affected by disturbances such as the movement of the operator OP. Therefore, it is possible to obtain the map data 73 reflecting the positions of the top plate 20 and the holder 22 while suppressing the influence of the disturbance. The target position TP can be reached on an appropriate driving path such as a shortest path or a path avoiding an obstacle. In addition, as in EP4330913A, the map data 73 can be easily updated as compared to updating the map data according to the output data of the LiDAR sensor by using the output data of the optical sensor and the radar sensor. Further, the map data 73 reflecting the real-time position of the component such as the top plate 20 can be obtained.

As shown in FIG. 8, the self-position estimation/map data creation unit 62 estimates the driving amount of the carriage unit 16 based on the change amount of the position of the same feature point FP included in the feature point extraction result 71PF of the previous frame and the feature point extraction result 71CF of the current frame. Therefore, it is possible to contribute to the improvement of the estimation accuracy of the self-position PS.

As shown in FIG. 9, the self-position estimation/map data creation unit 62 recognizes the top plate 20 and the holder 22 during movement based on the top plate position detection data 74 and the holder position detection data 75, and estimates the driving amount by excluding the feature point FP related to the top plate 20 and the holder 22 recognized as being in movement. The feature point FP related to the top plate 20 and the holder 22 during movement is noise in the estimation of the driving amount. Therefore, by excluding the feature point FP related to the top plate 20 and the holder 22 during movement from the estimation of the driving amount, it is possible to further contribute to the improvement of the estimation accuracy of the self-position PS.

As shown in FIG. 7 and the like, the map data 73 is three-dimensional data. Therefore, the map data 73 including the structure having a height and changing the height position, such as the decubitus imaging table 19 and the upright imaging table 21, can be created. The target position TP can be reached on an appropriate driving path such as a shortest path or a path avoiding an obstacle.

As shown in FIG. 11, the map data 73 is set to have a lower spatial resolution in the height direction than in the horizontal direction. Therefore, information on the horizontal surrounding environment that is important for the autonomous driving can be included in the map data 73. In addition, a load of the process of creating the map data 73 can be reduced.

As shown in FIGS. 1 and 2, the structure includes the decubitus imaging table 19 and the upright imaging table 21, and the component is the top plate 20 of the decubitus imaging table 19 and the holder 22 of the upright imaging table 21. The decubitus imaging table 19 and the upright imaging table 21 are installed in the imaging room RM in almost all cases. In most cases, the top plate 20 and the holder 22 are movable. Therefore, by setting the structure to the decubitus imaging table 19 and the upright imaging table 21 and setting the component to the top plate 20 and the holder 22, the map data 73 that fits the configuration of the general imaging room RM can be created.

### Second Embodiment

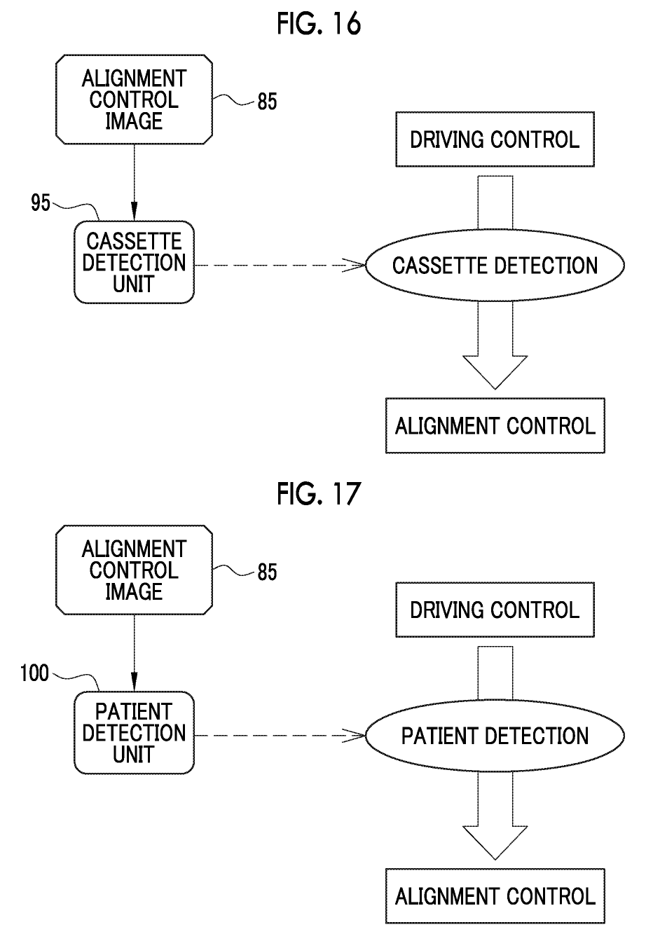

In the first embodiment, the driving control is transitioned to the alignment control in a case in which the self-position PS is the target position TP, but the present disclosure is not limited to this. As an example, the second embodiment may be as shown in FIGS. 16 and 17.

As shown in FIG. 16, the processor 40 of the present embodiment functions as a cassette detection unit 95 in addition to each processing unit of the first embodiment. The alignment control image 85 is input to the cassette detection unit 95. The cassette detection unit 95 detects whether or not the electronic cassette 12 is shown in the alignment control image 85 by using an image recognition technology.

In a case in which it is detected by the cassette detection unit 95 that the electronic cassette 12 is shown in the alignment control image 85, the processor 40 of the present embodiment transitions from the driving control to the alignment control. The cassette detection unit 95 may detect whether or not the electronic cassette 12 is shown in the alignment control image 85 by using the trained model such as the cassette contour extraction model 86 of the first embodiment.

Alternatively, as shown in FIG. 17, the processor 40 of the present embodiment functions as a patient detection unit 100 in addition to each processing unit of the first embodiment. The alignment control image 85 is input to the patient detection unit 100. The patient detection unit 100 detects whether or not the patient P is shown in the alignment control image 85 by using an image recognition technology. In a case in which it is detected by the patient detection unit 100 that the patient P is shown in the alignment control image 85, the processor 40 of the present embodiment transitions from the driving control to the alignment control. In a case in which it is detected that the electronic cassette 12 or the patient P is shown in the alignment control image 85 in this way, the driving control is transitioned to the alignment control in the same manner as in a case in which the self-position PS is the target position TP, the driving control can be transitioned to the alignment control at a good timing, and the alignment control can be executed without a hitch.

### Third Embodiment

In the first embodiment, the contour OLC of the electronic cassette 12 is extracted from the alignment control image 85, but the present disclosure is not limited thereto. As an example, the third embodiment may be as shown in FIG. 18.

In FIG. 18, the processor 40 of the present embodiment functions as a joint point extraction unit 105 instead of the cassette contour extraction unit 80 of the first embodiment. The alignment control image 85 is sequentially input to the joint point extraction unit 105. The joint point extraction unit 105 extracts the joint point J of the patient P from the alignment control image 85 by using a joint point extraction model 106. FIG. 18 shows a case in which both shoulder joints, both elbow joints, and both hip joints are extracted as the joint points J. According to the line connecting each joint point J, the center IC of the imaging part of the patient P and the posture of the patient P with respect to the radiation source 13 are known. The joint point extraction model 106 is a trained model configured by, for example, a convolutional neural network, similarly to the cassette contour extraction model 86. The joint point extraction unit 105 outputs a joint point extraction result 107 to the alignment control unit 82.

In a case in which the posture of the radiation source 13 is inclined with respect to the patient P, the alignment control unit 82 controls the driving of the alignment actuator 52 to rotate the radiation source 13 to eliminate the inclination. In addition, in a case where the irradiation center RC of the radiation R and the center IC of the imaging part of the patient P are shifted, the alignment control unit 82 controls the driving of the alignment actuator 52 to move the radiation source 13 to eliminate the shift. By extracting the joint point J of the patient P from the alignment control image 85 in this way, it is possible to contribute to the higher-accuracy alignment between the radiation source 13 and the electronic cassette 12.

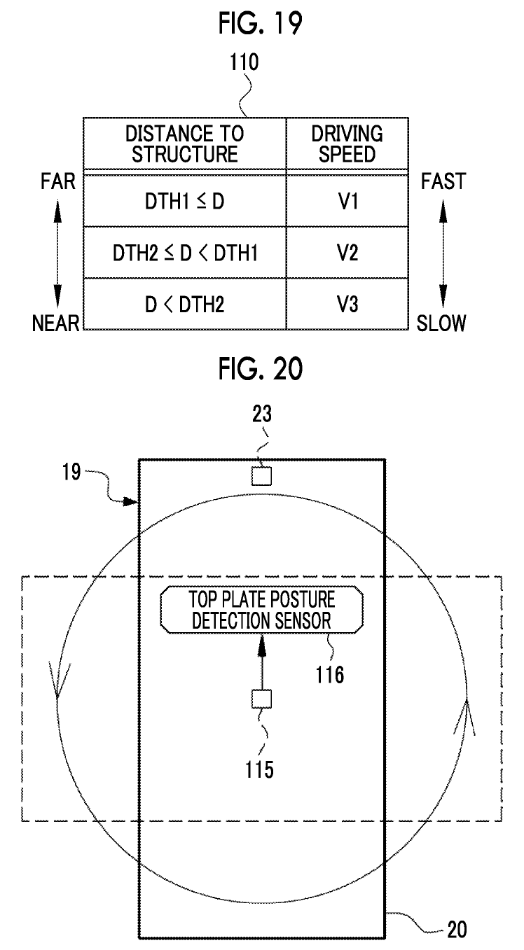

As shown in Table 110 in FIG. 19 as an example, the driving control unit 63 may change the driving speed of the carriage unit 16 according to the distance to the structure obtained from the estimated self-position PS and the map data 73. Specifically, the driving control unit 63 increases the degree of reduction in the driving speed as the distance to the structure is shorter. That is, in a case in which the distance D to the structure is equal to or longer than the first distance threshold value DTH1 (DTH1 ≤ D), the driving control unit 63 causes the carriage unit 16 to drive at the driving speed V1. In a case in which the distance D to the structure is equal to or longer than the second distance threshold value DTH2 and shorter than the first distance threshold value DTH1 (DTH2 ≤ D < DTH1), the driving control unit 63 causes the carriage unit 16 to drive at the driving speed V2 that is slower than the driving speed V1. In a case in which the distance D to the structure is shorter than the second distance threshold value DTH2 (D < DTH2), the driving control unit 63 causes the carriage unit 16 to drive at the driving speed V3 that is slower than the driving speed V2. The first distance threshold value DTH1 is, for example, 1 m, and the second distance threshold value DTH2 is, for example, 30 cm. The driving speed V2 is, for example, 1/2 of the driving speed V1, and the driving speed V3 is, for example, 1/10 of the driving speed V1.

In this way, it is possible to have a time margin to retreat the structure or change the driving path to avoid the structure in a case in which the radiation generation apparatus 11 is likely to collide with the structure. In addition, even in a case in which the radiation generation apparatus 11 collides with the structure, the impact can be alleviated. The safety of the operator OP and the patient P can also be ensured.

As shown in FIG. 20 as an example, the top plate 20 of the decubitus imaging table 19 may be rotated around a vertical axis. In this case, in addition to the top plate position detection sensor 23, a top plate posture detection sensor 115 is provided on the top plate 20. The top plate posture detection sensor 115 detects the posture (rotation position) of the top plate 20. The top plate posture detection sensor 115 is, for example, a rotary encoder, a potentiometer, a gyro sensor, or a combination of a plurality of types of these sensors. The top plate posture detection sensor 115 is an example of a "detection sensor" according to the disclosed technology. In this case, the self-position estimation/map data creation unit 62 updates the map data 73 by using the top plate posture detection data 116 from the top plate posture detection sensor 115 in addition to the top plate position detection data 74 and the like.

Although the decubitus imaging table 19 and the upright imaging table 21 are exemplified as the structure and the top plate 20 and the holder 22 are exemplified as the component, the present disclosure is not limited to this. The structure may be a patient support. The patient support is a support column with a handrail in a case of the upright imaging of the patient P. The component in this case is a handle that is rotated about the support column between the accommodation position and the use position. In addition, the structure may be a ceiling-mounted display. The ceiling-mounted display is composed of an arm suspended from a ceiling and a display attached to a distal end of the arm. The component in this case is a display of which the position and the posture can be changed by the arm.

The map data 73 may be updated only in a case in which the component is likely to be an obstacle to the autonomous driving in the position and/or the posture. The map data 73 may be updated, that is, the map data 73 may be updated at regular intervals in a case in which a predetermined time (for example, 30 seconds to 1 minute) has elapsed since the previous update of the map data 73. For example, a camera that images the entire imaging room RM is provided, and the amount of movement of the operator OP, the patient P, the top plate of the decubitus imaging table 19, the holder 22 of the upright imaging table 21, or the like that can be a disturbance is detected from an image of the camera. Then, the map data 73 may be updated in a case in which the detected amount of movement is equal to or more than a predetermined amount.

The alignment control image 85 may be displayed on the operation panel 29. In this case, the contour OLC of the electronic cassette 12, the center CC of the detection surface 17 of the electronic cassette 12, the irradiation center RC of the radiation R, and the like may be superimposed and displayed on the alignment control image 85 as marks.

The carriage unit 16 may be omitted, and the wheels 15 may be directly attached to a lower portion of the body part 14.

The camera 32 may be attached to the arm 28 instead of the radiation source 13. Similarly, the camera 33 may be provided in the body part 14 instead of the carriage unit 16. A plurality of the cameras 32 and 33 may be provided.

The camera 32 and the camera 33 may be integrated into one camera. In this case, the orientation of the camera is set to an orientation in which the driving control image 70 can be captured in a case where the driving control is executed, and is set to an orientation in which the alignment control image 85 can be captured in a case where the alignment control is executed.

The environment information sensor is not limited to the example of the camera 33. A light detection and ranging (LiDAR) sensor, a time-of-flight (TOF) sensor, or the like may be used. In addition, an inertial measurement unit (IMU) in which an acceleration sensor and a gyro sensor are combined, an ultrasonic sensor, a radar sensor, a magnetic sensor, or the like may be used.

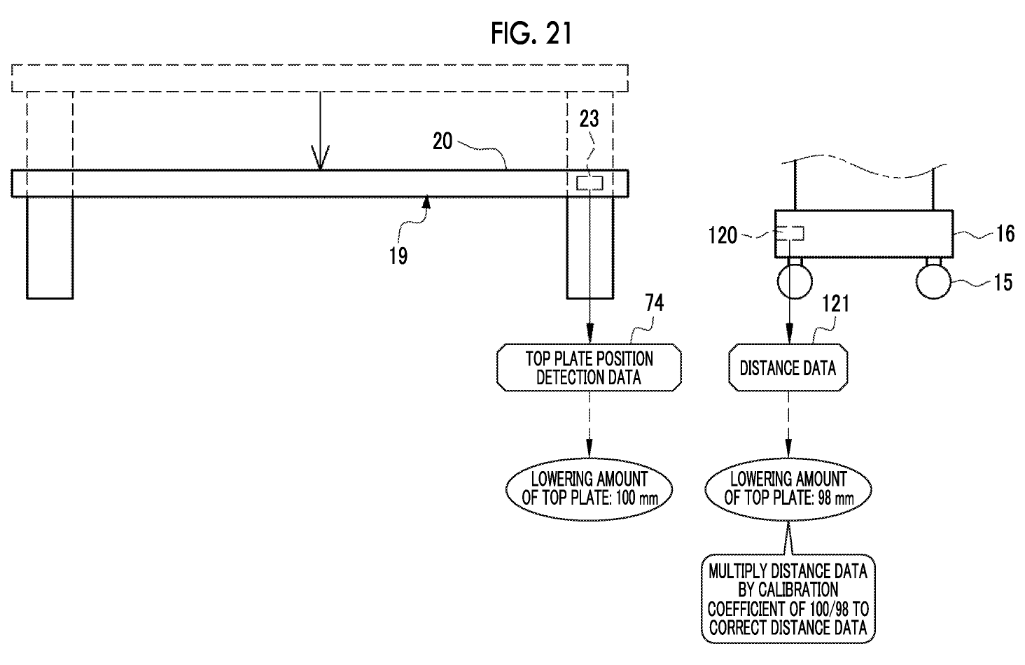

A case in which the environment information sensor is a distance sensor 120 such as a LiDAR sensor or a time-of-flight (TOF) sensor instead of the camera 33 will be considered. In this case, as shown in FIG. 21 as an example, the radiation generation apparatus 11 may comprise a calibration mode of correcting distance data 121 output from the distance sensor 120 by using a displacement amount of the component based on the detection data. In the calibration mode, the component is moved by a predetermined amount, and the displacement amount of the component is measured by the detection sensor and the distance sensor 120.

FIG. 21 shows a case in which the top plate 20 is lowered by a predetermined amount of 100 mm. Then, a case is shown in which the lowering amount of the top plate 20 based on the top plate position detection data 74 output from the top plate position detection sensor 23 is 100 mm, and the lowering amount of the top plate 20 based on the distance data 121 output from the distance sensor 120 is 98 mm. In this case, the self-position estimation/map data creation unit 62 multiplies the distance data 121 by a calibration coefficient of 100/98 to correct the distance data 121. The displacement amount of the component based on the detection data is more accurate than the displacement amount of the component based on the distance data 121. Therefore, the more accurate distance data 121 can be obtained, and as a result, the more accurate estimation of the self-position and the creation of the map data 73 can be performed. In order to facilitate the measurement of the displacement amount of the component by the distance sensor 120, a marker may be provided on the component.

The radiographic image detection device is not limited to the example of the electronic cassette 12. A computed radiography (CR) cassette may be used. In addition, the subject is not limited to the example of the patient P. The subject may be a diseased animal such as a dog or a cat.

In each of the above-described embodiments, for example, each process of each processing unit such as the image acquisition unit 60, the feature point extraction unit 61, the self-position estimation/map data creation unit 62, the driving control unit 63, the cassette contour extraction unit 80, the radiation source position/posture derivation unit 81, the alignment control unit 82, the cassette detection unit 95, the patient detection unit 100, and the joint point extraction unit 105 is executed by any computer. In addition, any computer may execute these types of processing by a processor as hardware, a program as software, or a combination thereof. In such a case, the processor is configured to execute various types of processing in each of the above-described embodiments in cooperation with the program, and may function as each unit or each means in each of the above-described embodiments. In addition, the execution order of the processing by the processor is not limited to the above-described order and may be changed as appropriate. Any computer may be a general-purpose computer, a computer for specific use, a workstation, or another system capable of executing each processing.

The processor may be configured using one or more pieces of hardware, and the type of hardware is not limited. For example, the processor may be configured by an example of the CPU or a micro processing unit (MPU), a programmable logic device such as a field programmable gate array (FPGA), a dedicated circuit for executing specific processing such as an application specific integrated circuit (ASIC), a graphics processing unit (GPU), a neural processing unit (NPU), or the like. The types of hardware may be a combination of different types of hardware. In a case in which the plurality of types of hardware are configured to execute one or a plurality of types of processing of a certain processor, the plurality of types of hardware may exist in devices physically separated from each other or may exist in the same device. Furthermore, in any of the embodiments, the order of each processing performed by the processor is not limited to the above-described order, and may be changed as appropriate. The hardware is configured by an electric circuit (circuitry) in which circuit elements such as semiconductor elements are combined.

The program may be software such as firmware or a microcode. Furthermore, the program may be, for example, a program module group, and each function thereof may be implemented by a processor configured to execute each function. The program may be a program code or a plurality of code segments stored in one or a plurality of non-transitory computer-readable media (for example, a storage medium or other storage). The program may be stored in the plurality of non-transitory computer-readable media existing in physically separated devices. The program code or the code segment may represent any combination of procedures, functions, subprograms, routines, subroutines, modules, software packages, classes, instructions, data structures, or program statements. The program code or the code segments may be connected to other code segments or hardware circuits by transmitting and receiving information, data, an argument, a parameter, or content of a memory.

The technologies according to the following supplementary notes can be understood from the above description.

### Supplementary Note 1

A radiation generation apparatus, in which a body part including a radiation source that emits radiation toward a subject is autonomously drivable by a driving mechanism having wheels, the radiation generation apparatus comprising:

an environment information sensor; and

a processor,

wherein the processor is configured to:

create map data of a surrounding environment while estimating a self-position from output data of the environment information sensor;

acquire detection data of a detection sensor that is provided in a structure in the surrounding environment and that detects a position and/or a posture of a movable component of the structure; and

update the map data by using the detection data.

### Supplementary Note 2

The radiation generation apparatus according to Supplementary Note 1,

wherein the processor is configured to estimate a driving amount of the driving mechanism based on a change amount of a position of the same structure included in past output data and current output data.

### Supplementary Note 3

The radiation generation apparatus according to Supplementary Note 2,

wherein the processor is configured to:

recognize the component during movement based on the detection data; and

estimate the driving amount by excluding the component recognized as being in movement.

### Supplementary Note 4

The radiation generation apparatus according to any one of Supplementary Notes 1 to 3,

wherein the map data is three-dimensional data.

### Supplementary Note 5

The radiation generation apparatus according to Supplementary Note 4,

wherein the map data is set to have a lower spatial resolution in a height direction than in a horizontal direction.

### Supplementary Note 6

The radiation generation apparatus according to any one of Supplementary Notes 1 to 5,

wherein the processor is configured to change a driving speed of the driving mechanism according to a distance to the structure.

### Supplementary Note 7

The radiation generation apparatus according to Supplementary Note 6,

wherein the processor is configured to increase a degree of reduction in the driving speed as the distance to the structure is shorter.

### Supplementary Note 8

The radiation generation apparatus according to any one of Supplementary Notes 1 to 7,

wherein the environment information sensor includes a distance sensor, and the output data includes distance data, and

the processor is configured to correct the distance data by using a displacement amount of the component based on the detection data.

### Supplementary Note 9

The radiation generation apparatus according to any one of Supplementary Notes 1 to 8,

wherein the structure includes a decubitus imaging table, and the component is a top plate of the decubitus imaging table.

### Supplementary Note 10

The radiation generation apparatus according to any one of Supplementary Notes 1 to 9,

wherein the structure includes an upright imaging table, and the component is a holder of the upright imaging table.

The technology of the present disclosure can also be combined with various embodiments and/or various modification examples described above, as appropriate. In addition, it goes without saying that the present disclosure is not limited to each of the embodiments described above, various configurations can be adopted as long as the configuration does not deviate from the gist. Furthermore, the technology of the present disclosure extends to a storage medium that non-transitorily stores the program, and a computer program product including the program, in addition to the program.

The above-described contents and the above-shown contents are the detailed description of the parts according to the technology of the present disclosure, and are merely an example of the technology of the present disclosure. For example, the above description of the configuration, the function, the operation, and the effect are the description of examples of the configuration, the function, the operation, and the effect of the parts according to the technology of the present disclosure. Accordingly, it goes without saying that unnecessary parts may be deleted, new elements may be added, or replacements may be made with respect to the above-described contents and the above-shown contents within a range that does not deviate from the gist of the technology of the present disclosure. In addition, in order to avoid complications and facilitate grasping the parts according to the technology of the present disclosure, in the above-described contents and the above-shown contents, the description of technical general knowledge and the like that do not particularly require description for enabling the implementation of the technology of the present disclosure are omitted.

In the present specification, "A and/or B" has the same meaning as "at least one of A or B". That is, "A and/or B" means that it may be only A, only B, or a combination of A and B. In addition, in the present specification, also in a case where three or more matters are expressed in association by "and/or", the same concept as "A and/or B" is applied.

All of the documents, the patent applications, and the technical standards described in the present specification are incorporated herein by reference to the same extent as in a case where each of the documents, patent applications, and technical standards is specifically and individually described by being incorporated by reference.

What is claimed is:

1. A radiation generation apparatus, in which a body part including a radiation source that emits radiation toward a subject is autonomously drivable by a driving mechanism having wheels, the radiation generation apparatus comprising:
an environment information sensor; and
a processor,
wherein the processor is configured to:
create map data of a surrounding environment while estimating a self-position from output data of the environment information sensor;
acquire detection data of a detection sensor that is provided in a structure in the surrounding environment and that detects a position and/or a posture of a movable component of the structure; and
update the map data by using the detection data.

2. The radiation generation apparatus according to claim 1,
wherein the processor is configured to estimate a driving amount of the driving mechanism based on a change amount of a position of the same structure included in past output data and current output data.

3. The radiation generation apparatus according to claim 2,
wherein the processor is configured to:
recognize the component during movement based on the detection data; and
estimate the driving amount by excluding the component recognized as being in movement.

4. The radiation generation apparatus according to claim 1,
wherein the map data is three-dimensional data.

5. The radiation generation apparatus according to claim 4,
wherein the map data is set to have a lower spatial resolution in a height direction than in a horizontal direction.

6. The radiation generation apparatus according to claim 1,
wherein the processor is configured to change a driving speed of the driving mechanism according to a distance to the structure.

7. The radiation generation apparatus according to claim 6,
wherein the processor is configured to increase a degree of reduction in the driving speed as the distance to the structure is shorter.

8. The radiation generation apparatus according to claim 1,
wherein the environment information sensor includes a distance sensor, and the output data includes distance data, and
the processor is configured to correct the distance data by using a displacement amount of the component based on the detection data.

9. The radiation generation apparatus according to claim 1,
wherein the structure includes a decubitus imaging table, and the component is a top plate of the decubitus imaging table.

10. The radiation generation apparatus according to claim 1,
wherein the structure includes an upright imaging table, and the component is a holder of the upright imaging table.

11. An operation method of a radiation generation apparatus, in which a body part including a radiation source that emits radiation toward a subject is autonomously drivable by a driving mechanism having wheels, the operation method comprising:
creating map data of a surrounding environment while estimating a self-position from output data of an environment information sensor;
acquiring detection data of a detection sensor that is provided in a structure in the surrounding environment and that detects a position and/or a posture of a movable component of the structure; and
updating the map data by using the detection data.

12. A non-transitory computer-readable storage medium storing an operation program of a radiation generation apparatus, in which a body part including a radiation source that emits radiation toward a subject is autonomously drivable by a driving mechanism having wheels, the operation program causing a computer to execute a process comprising:
creating map data of a surrounding environment while estimating a self-position from output data of an environment information sensor;
acquiring detection data of a detection sensor that is provided in a structure in the surrounding environment and that detects a position and/or a posture of a movable component of the structure; and
updating the map data by using the detection data.
